(19) United States
(12) Patent Application Publication
(10) Pub. No.: US 2010/0152779 A1
(43) Pub. Date: Jun. 17, 2010

(54) INTER-TRANSVERSE PROCESS SPACER DEVICE AND METHOD FOR USE IN CORRECTING A SPINAL DEFORMITY

(75) Inventors: Randall N. Allard, Issaquah, WA (US); Kent M. Anderson, Sunnyvale, CA (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/712,719

(22) Filed: Feb. 25, 2010

Related U.S. Application Data
(62) Division of application No. 11/559,938, filed on Nov. 15, 2006.

(51) Int. Cl. A61B 17/88

(52) U.S. Cl. 606/279

(57) ABSTRACT

An inter-transverse process spacer device for placement between two adjacent transverse processes, includes a spacer member with deformable first and second ends and may include a connector. The inter-transverse process spacer device may also include a flexible, fillable container for containing an injectable material that is compressible following implantation. The container is impermeable to the material it will be filled with. A structural mesh, for example, made of PET fabric and interwoven shape-memory alloy wire, provides structure for and containment of the container, as well as shape control of the inter-transverse process spacer device. The material can be injected into the container through a conduit. The inter-transverse process spacer device is sized and configured to allow for placement between adjacent transverse processes to produce a lateral force for correcting a spinal deformity. A method for correcting a spinal deformity using the inter-transverse process spacer device is also disclosed.

### CROSS-REFERENCE TO RELATED APPLICATIONS

This application is a divisional of co-pending U.S. application Ser. No. 11/559,938, filed Nov. 15, 2006, the entire contents of which are hereby incorporated by reference.

This application contains subject matter which is related to the subject matter of the following applications, which are hereby incorporated herein by reference in their entirety:

“Surgical Spacer,” by Lange et al., U.S. Ser. No. 11/438,940, filed May 23, 2006;

“Surgical Spacer with Shape Control,” by Lange et al., U.S. Ser. No. 11/438,891, filed May 23, 2006;

“Implants and Methods for Inter-Transverse Process Dynamic Stabilization of a Spinal Segment,” by Brumeau et al., U.S. Ser. No. 11/104,267, filed Apr. 12, 2005; and

“Intercostal Spacer Device and Method for Use in Correcting Spinal Deformity,” by Stoklund et al., U.S. Ser. No. 11/470,810, filed Sep. 7, 2006.

### TECHNICAL FIELD

The present invention relates generally to orthopaedic implants used for the correction of spinal deformities, and more specifically, but not exclusively, concerns apparatuses placed between the transverse processes of two adjacent vertebral bodies to allow for deformity correction or healing of the spinal column.

### BACKGROUND OF THE INVENTION

To secure and treat spinal deformities, including scoliosis, it is a generally accepted practice to place implants adjacent to or into the vertebrae to produce loads,for correcting an abnormal curvature of the spine and to maintain appropriate vertebral support for the healing of the implanted bone fusion material.

Typically, for treatment of scoliosis and lateral stenosis, spinal implant systems are implanted through a posterior approach to the spinal column and utilize a rod or cable as the support and stabilizing element connected to a series of two or more bone fasteners that have been inserted into two or more vertebrae. The connections between these components are then secured, thereby fixing a supporting and spine straighting force construct to multiple levels in the spinal column.

### SUMMARY OF THE INVENTION

Advancement of the state of orthopaedic implants and the treatment of pediatric and adolescent scoliosis is believed to be desirable. The present invention satisfies the need for improvements to the surgical treatment by providing a more mechanically efficient and minimally invasive inter-transverse process spacer device for implantation between the transverse processes of multiple vertebral levels within a patient's spinal column. The inter-transverse process spacer device is a one piece construct fabricated from a biocompatible material. Alternatively, the inter-transverse process spacer device may be a multiple piece construct that includes a flexible container that is fillable in situ to a desired amount, with a structure associated with at least part of the container providing shape control of the-inter-transverse process spacer device. An optional conduit coupled to the container allows for filling of the container, for example, by injecting a material into the container following placement of the container in situ.

The present invention provides in one aspect, an inter-transverse process spacer device. The inter-transverse process spacer device includes a spacer member that has a superior end and an inferior end. The spacer member is sized and configured to enable placement between two adjacent transverse processes, allowing the inter-transverse process spacer device to resist dislodgement from between the two adjacent transverse processes and produce a force for correcting a spinal deformity.

The present invention provides in another aspect, an inter-transverse process spacer device that includes a flexible container for receiving an injectable material that is compressible following implantation between two adjacent transverse processes, wherein the flexible container is substantially impermeable to the injectable material. The inter-transverse process spacer device fiuther includes a conduit coupled to the flexible container for delivering the injectable material, and a structure that is associated with at least part of the flexible container for controlling part of the shape of the inter-transverse process spacer device and containing the material, the structure having a shape to fit between two adjacent transverse processes.

The present invention provides in another aspect, a method for correcting a spinal deformity. The method includes the step of obtaining at least one inter-transverse process spacer device, the inter-transverse process spacer device includes a spacer member having first and second ends, the spacer member being sized for placement between a first transverse process and an adjacent second transverse process of a patient. The method further includes the positioning of the at least one inter-transverse process spacer device between the two adjacent transverse processes of the patient, producing a force to correct the spinal deformity of the patient.

Another aspect of the present invention provides a method of correcting a spinal deformity. The method includes obtaining an inter-transverse process spacer device, the inter-transverse process spacer device includes a flexible container for containing an injectable material that is compressible following implantation and is substantially impermeable to the injectable material. The inter-transverse process spacer device further includes a conduit attached to the flexible container for delivering the injectable material, and a structure associated with at least part of the flexible container, the structure has a shape of the inter-transverse process spacer device that is sized and configured to fit between adjacent transverse processes in a patient. The method further includes positioning the inter-transverse process spacer device between two adjacent transverse processes. The injectable material is then injected into the flexible container through the conduit such that the shape of the structure is achieved, thus producing a force to correct the spinal deformity of the patient.

Further, additional features and advantages are realized through the techniques of the present invention. Other embodiments and aspects of the invention are described in detail herein and are considered a part of the claimed invention.

### BRIEF DESCRIPTION OF THE DRAWINGS

The subject matter which is regarded as the invention is particularly pointed out and distinctly claimed in the claims at the conclusion of the specification. The foregoing and other objects, features and advantages of the invention are apparent from the following detailed description taken in conjunction with the accompanying drawings in which:

FIG. 1A is a perspective view of one embodiment of an inter-transverse process spacer device configured as a generally rectangular shape, shown before being implanted between two adjacent transverse processes, in accordance with an aspect of the present invention;

FIG. 1B is a perspective view of one embodiment of an inter-transverse process spacer device configured as a generally cylindrical shape, shown before being implanted between two adjacent transverse processes, in accordance with an aspect of the present invention;

FIG. 1C is a posterior elevational view of the inter-transverse process spacer device embodiment of FIG. 1A, implanted between two transverse processes with a single connector, in accordance with an aspect of the present invention;

FIG. 1D is a side, cross-sectional elevational view of the inter-transverse process spacer device embodiment of FIG. 1A with two single connectors shown passing through a spacer member and disposed between the cross-section of two adjacent transverse processes, in accordance with an aspect of the present invention;

FIG. 1E is a side, cross-sectional elevational view of the inter-transverse process spacer device embodiment of FIG. 1A, taken along line 1E-1E, shown disposed between the cross-section of two adjacent transverse processes, with a single connector surrounding the entire inter-transverse process spacer device, in accordance with an aspect of the present invention;

FIG. 1F is a side, cross-sectional elevational view of the inter-transverse process spacer device embodiment of FIG. 1A, shown disposed between the cross-section of two adjacent transverse processes, with a single connector utilizing an alternative securing configuration, in accordance with an aspect of the present invention;

FIG. 1G is a side, cross-sectional elevational view of the inter-transverse process spacer device embodiment of FIG. 1A, shown disposed between the cross-section of two adjacent transverse processes, with a single connector secured with channels positioned on the anterior and posterior external sides, in accordance with an aspect of the present invention;

FIG. 1H is a side, cross-sectional view of the inter-transverse process spacer device embodiment of FIG. 1A, shown disposed between the cross-section of two adjacent transverse processes with tong structures disposed near the superior end and inferior end of the spacer member, in accordance with an aspect of the present invention.

FIG. 2A is a side, cross-sectional view of one embodiment of an inter-transverse process spacer device showing the inner and outer portions before compressive loads are applied between two adjacent transverse processes, in accordance with an aspect of the present invention;

FIG. 2B is a side, cross-sectional elevational view of the inter-transverse process spacer device embodiment of FIG. 2A following the application of compressive loads between two adjacent transverse processes, in accordance with an aspect of the present invention;

FIG. 3A is a side elevational view of one embodiment of an inter-transverse process spacer device shown disposed between the cross-section of two adjacent transverse processes, in accordance with an aspect of the present invention;

FIG. 3B is a side elevational view of the inter-transverse process spacer device embodiment of FIG. 3A with two single connectors shown disposed between the cross-section of two adjacent transverse processes, in accordance with an aspect of the present invention;

FIG. 3C is a side elevational view of the inter-transverse process spacer device embodiment of FIG. 3A, shown disposed between the cross-section of two adjacent transverse processes, with a single connector surrounding the entire inter-transverse process spacer device, in accordance with an aspect of the present invention;

FIG. 3D is a side elevational view of the inter-transverse process spacer device embodiment of FIG. 3A, shown disposed between the cross-section of two adjacent transverse processes, with a single connector utilizing an alternative securing configuration, in accordance with an aspect of the present invention;

FIG. 3E is a perspective view of the inter-transverse process spacer device embodiment of FIG. 3A, shown implanted between two adjacent transverse processes and an offset single connector, in accordance with an aspect of the present invention;

FIG. 3F is a side elevational view of the inter-transverse process spacer device embodiment of FIG. 3A, shown disposed between the cross-section of two adjacent transverse processes, with two alternative single connectors inserted through two bore holes, in accordance with an aspect of the present invention;

FIG. 3G is a perspective view of the inter-transverse process spacer device embodiment of FIG. 3F with the two alternative single connectors extracted from the two bore holes, in accordance with an aspect of the present invention;

FIG. 3H is a side elevational view of the inter-transverse process spacer device embodiment of FIG. 3A, shown disposed between the cross-section of two adjacent transverse processes with tong structures disposed on the superior portion and inferior portion of the superior pair of anus and inferior pair of anus, respectively, in accordance with an aspect of the present invention;

FIG. 31 is a posterior-lateral, perspective view of one embodiment of an inter-transverse process spacer system shown disposed between three adjacent transverse processes, in accordance with an aspect of the present invention;

FIG. 4A is a perspective view of one embodiment of an inter-transverse process spacer device, in accordance with an aspect of the present invention;

FIG. 4B is a posterior-lateral perspective view of one embodiment of an inter-transverse process spacer system shown disposed between three adjacent transverse processes, in accordance with an aspect of the present invention;

FIG. 5A is a perspective view of one embodiment of an inter-transverse process spacer device, in accordance with an aspect of the present invention;

FIG. 5B is a posterior-lateral, perspective elevational view of one embodiment of an inter-transverse process spacer device system shown disposed between three adjacent transverse processes, in accordance with an aspect of the present invention;

FIG. 6 is a perspective view of one embodiment of an inter-transverse process spacer device, in accordance with an aspect of the present invention;

FIG. 7A is a side elevational view of one embodiment of an inter-transverse process spacer device, in accordance with an aspect of the present invention;

FIG. 7B is a lateral, elevational view of the inter-transverse process spacer device embodiment of FIG. 7A shown with slits expanded, and being moved in au posterior to anterior direction allowing inter-transverse spacer device to be positioned over two adjacent transverse processes, in accordance with an aspect of the present invention;

FIG. 8 is a posterior-lateral, perspective view of one embodiment of an inter-transverse process spacer device, in accordance with an aspect of the present invention;

FIG. 9A is a posterior-lateral, perspective view of one embodiment of an inter-transverse process spacer device shown with the bow apex positioned laterally, in accordance with an aspect of the present invention;

FIG. 9B is a posterior-lateral, perspective view of the inter-transverse process spacer device embodiment of FIG. 9A shown with the bow apex positioned medially, in accordance with an aspect of the present invention;

FIG. 10A is a posterior-lateral, perspective view of one embodiment of an inter-transverse process spacer device shown with tethers attached to a superior end and posterior end, with each tether being passed through a transverse hole in a transverse process, in accordance with an aspect of the present invention;

FIG. 10B is a posterior-lateral, perspective view of the inter-transverse process spacer device embodiment of FIG. 10A shown with tethers attached to a spacer member, with each tether connecting to a laterally-positioned ligamentus structure, in accordance with an aspect of the present invention

FIG. 11 is a lateral, elevational view of one embodiment of an inter-transverse process spacer device shown with angled biased surfaces located at the superior end and inferior end, contacting the outer cortex of two adjacent transverse processes, in accordance with an aspect of the present invention;

FIG. 12 is a perspective partial cut-away view of one embodiment of an unfilled inter-transverse process spacer device with the container in the structure, in accordance with an aspect of the present invention;

FIG. 13 is a posterior, elevational view of one embodiment of an inter-transverse process spacer device with an integrated container and structure, in accordance with an aspect of the present invention;

FIG. 14 is a cross-sectional elevational view of one embodiment of an inter-transverse process spacer device with an external container, in accordance with an aspect of the present invention; and

FIG. 15 depicts another embodiment of an inter-transverse process spacer device with an integrated container and structure, in accordance with another aspect of the present invention.

### BEST MODE FOR CARRYING OUT THE INVENTION

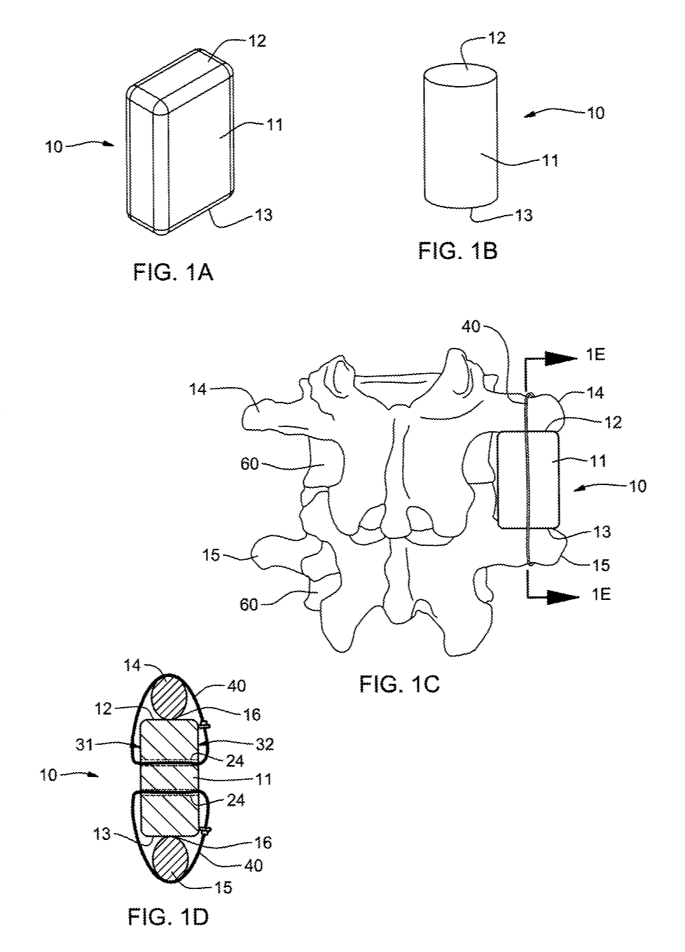

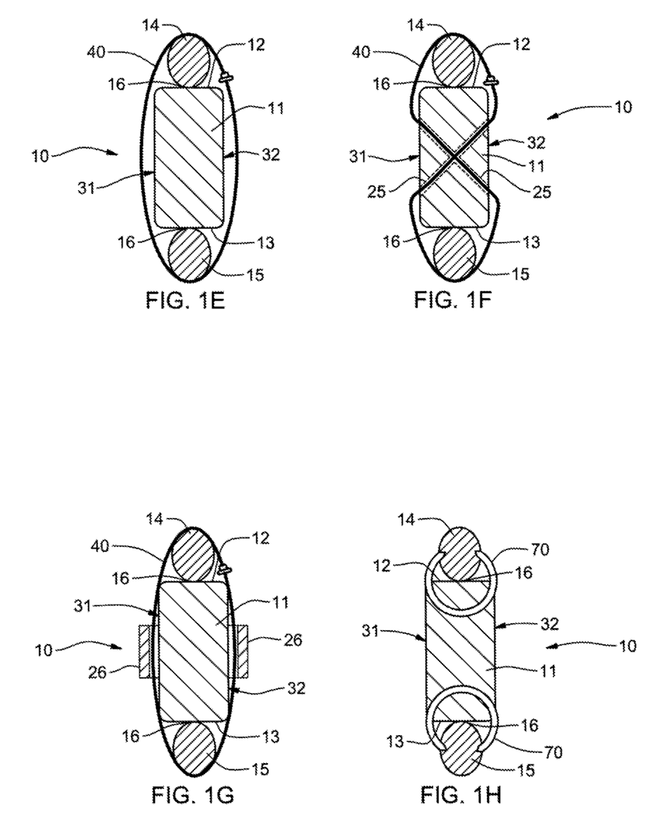

FIGS. 1A & 1B depict one embodiment of an inter-transverse process spacer device 10, in accordance with an aspect of the present invention. As shown, device 10 includes a spacer member 11 comprising a superior end 12 and an inferior end 13 with a central axis (not shown) extending between superior end 12 and inferior end 13. Spacer member 11 may be configured as a rectangular shape or as a cylindrical unitary body. Spacer member 11 may be fabricated from a material that allows superior end 12 and inferior end 13 to deform to the shape of a transverse process. Spacer member 11 is generally sized to be placed between two transverse processes 14, 15 (see FIG. 1C) and may be maneuvered in a manner to be positioned between two adjacent transverse processes 14, 15 causing the inferior aspect of the superior positioned transverse process 14 to contact superior end 12 and the superior aspect of the inferior positioned transverse process 15 to contact inferior end 13 resulting in the creation of depressions 16 on the surfaces of superior and inferior ends 12, 13. Depressions 16 will closely conform to the exterior surface of transverse processes 14, 15 and provide resistance to in vivo forces that may lead to dislodgement of spacer member 11 from its implanted position.

Typically, at least one through hole 24 is directed in the anterior to posterior direction and is located within spacer member 11 in the inter-transverse process spacer device 10. In one approach, connector 40 (see FIG. 1D) is inserted into hole 24 following the placement of inter-transverse process spacer device 10 between adjacent transverse processes 14, 15. As depicted in FIG. 1D, a first connector 40 may be inserted through passage or hole 24 that extends from an anterior surface 31 of spacer member 11 to a posterior surface 32 of spacer member and then wraps over the superior aspect of superior positioned transverse process 14 when in contact with superior end 12. A second connector 40 may be inserted through a second passage or hole 24 that is substantially parallel to the first connector 40 and also extends from anterior surface 31 to posterior surface 32 of spacer member 11. Second connector 40, after passing through hole 24, wraps over the inferior aspect of the inferior positioned transverse process 15. The ends of connectors 40 may be secured using crimps, knots, ties or other suitable fasteners. It should be understood to those skilled in the art that other securement techniques and configurations are contemplated and will depend on the type of connector 40 used with inter-transverse process spacer device 10.

As shown in FIG. 1E, an alternative method of securing inter-transverse process spacer device 10 between two adjacent transverse processes 14, 15 may include extending at least one connector 40 around the circumference of the exterior surface of spacer member 11 and the two adjacent transverse processes 14, 15. The ends of connector 40 may then be secured using crimps, knots, ties or other suitable fasteners, although it should be understood to those skilled in the art that other securement techniques and configurations are contemplated and will depend on the type of connector 40 used in securing inter-transverse process spacer device 10 between the two adjacent transverse processes 14, 15.

Yet another alternative method of securing inter-transverse process spacer device 10 between two adjacent transverse processes 14, 15 is shown in FIG. 1F. At least one connector 40 may be utilized in a generally figure-8 configuration by inserting multiple connectors 40 or a single connector 40 into an angled passage or hole 25 that extends from anterior surface 31 to posterior surface 32 of spacer member 11, with connector 40 being looped over the superior aspect of superior positioned transverse process 14. Connector 40 may be further passed through a second angled passage or hole 25 that extends from anterior surface 31 to posterior surface 32 of spacer member 11 allowing connector 40 to also loop over the inferior aspect of inferior positioned transverse process 15. The two ends of connector 40 may be secured using crimps, knots, ties or other suitable fastener. It should be understood to those skilled in the art that other securement techniques and configurations are contemplated and will depend on the type of connector 40 used within inter-transverse process spacer device 10.

A further alternative method of securing inter-transverse process spacer device 10 between two adjacent transverse processes 14, 15 is illustrated at FIG. 1G. At least one connector 40 extends around the circumference of the exterior surface of spacer member 11 and the two adjacent transverse processes 14, 15. Typically, disposed on at least anterior surface 31 is a channel or buckle structure 26 through which connector 40 is passed. It is contemplated that channel structure 26 may also be disposed on posterior surface 32, or alternatively on both anterior surface 31 and posterior surface 32. Channel structure 26 functions to facilitate keeping connector 40 aligned along the exterior surface of spacer niember 11. The ends of connector 40 may be secured using crimps, knots, ties or other suitable fasteners, although it should be understood to those skilled in the art that other securement techniques and configurations are contemplated and will depend on the type of connector 40 used in securing inter-transverse process spacer device 10 between the two adjacent transverse processes 14, 15.

Connector 40 may be in the form of a suture, wire, cable, tether, belt, band, cord or other suitable structure and may be, for example, fabricated from a material selected from the group consisting of carbon fiber composite polymers, bio-compatible metals, resorbable polymers, bio-inert polymeric materials, polyester, polyethylene, titanium, stainless steel and any combinations of these materials.

FIG. 1H depicts yet a further alternative method for securing inter-transverse process spacer device 10. A tong structure 70 may be disposed at superior end 12 and inferior end 13 with tong structure 70 being generally ring-shaped and functioning to pierce the anterior and posterior surfaces of transverse processes 14, 15. Alternatively, tong structure 70 may fix inter-transverse process spacer device 10 between two adjacent transverse processes 14, 15 by piercing surrounding soft tissue or ligamentus structures. Tong structure 70 may be fabricated from a deformable or springy material including, but not limited to PEEK, titanium, stainless steel, other bio-compatible metals, other bio-inert polymeric materials and any combinations of these materials.

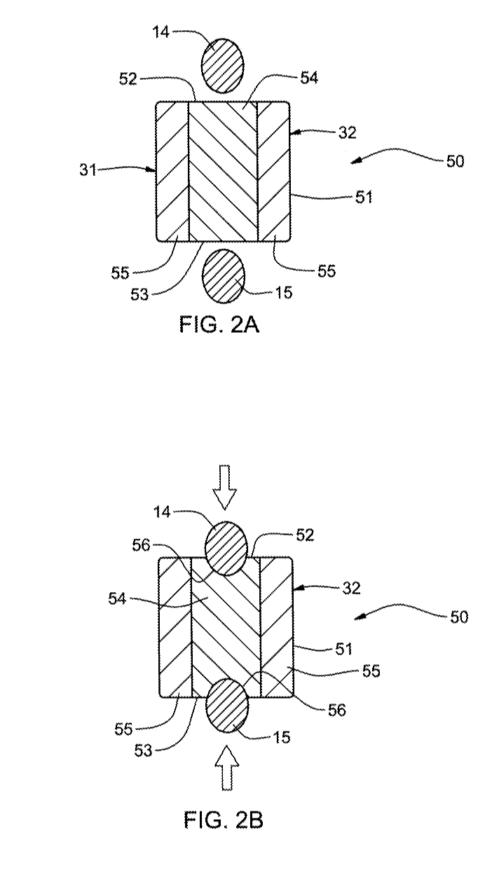

FIG. 2A shows an alternative embodiment of an inter-transverse process spacer device 50, in accordance with an aspect of the present invention, including a spacer member 51 comprising a superior end 52 and an inferior end 53 with a central axis (not shown) extending between superior end 52 and inferior end 53. Spacer member 51 further includes an inner portion 54 and an outer portion 55, inner portion 54 being fabricated from a material that is different than outer portion 55. The construct material for both inner portion 54 and outer portion 55 may be generally characterized as being deformable and elastic. The inner portion 54 is fabricated from a material that usually possesses a lower compression modulus in comparison to the material from which outer portion 55 is manufactured. The compression modulus for these two materials ranges between 0.003 and 4.2 GPa. Spacer member 51 is generally sized to be placed between two transverse processes 14, 15 and may be maneuvered in a manner to cause the inferior aspect of superior positioned transverse process 14 to contact superior end 52 and the superior aspect of inferior positioned transverse process 15 to contact inferior end 53. As seen in FIG. 2B, concavities 56 are formed in the surfaces located at superior and inferior ends 52, 53 of inner portion 54. The depth and shape of concavities 56 are dependent upon the magnitude of the in vivo forces applied by transverse processes 14, 15 following implantation. Typically, resultant concavities 56 will be shaped to closely conform to the exterior surface of transverse processes 14, 15 and thus, will assist in resisting post-operative forces that may lead to movement of spacer member 51 from its implanted position.

Although not shown, it should be understood to those skilled in the art that the various securement methods that have been described herein with connector 40 and tong structure 70 may also be utilized with inter-transverse process spacer device 50 to secure spacer member 51 between two adjacent transverse processes 14, 15.

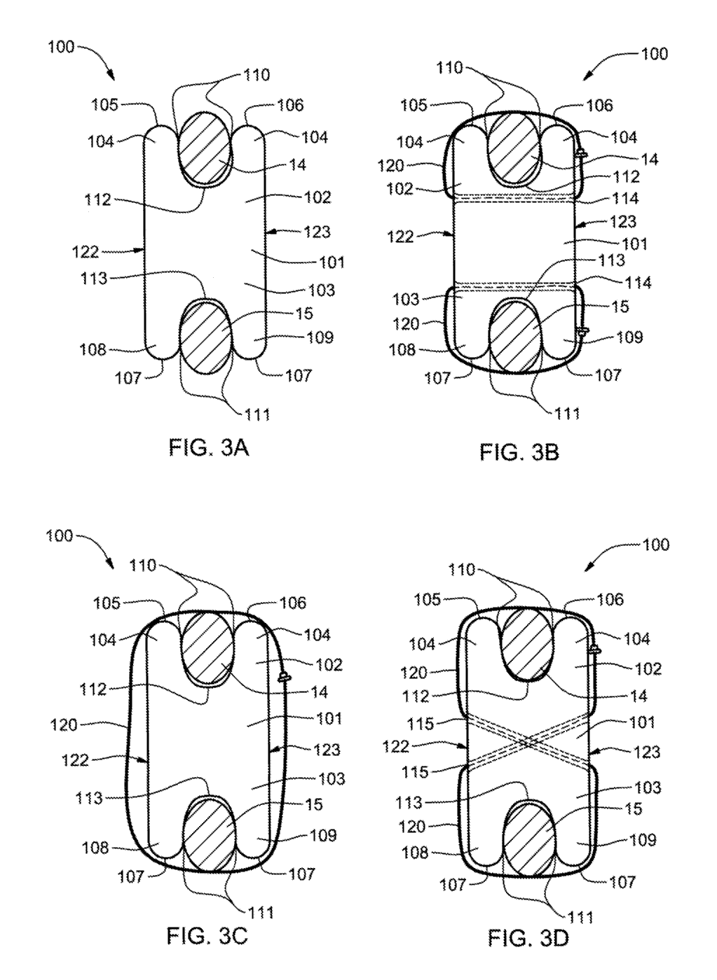

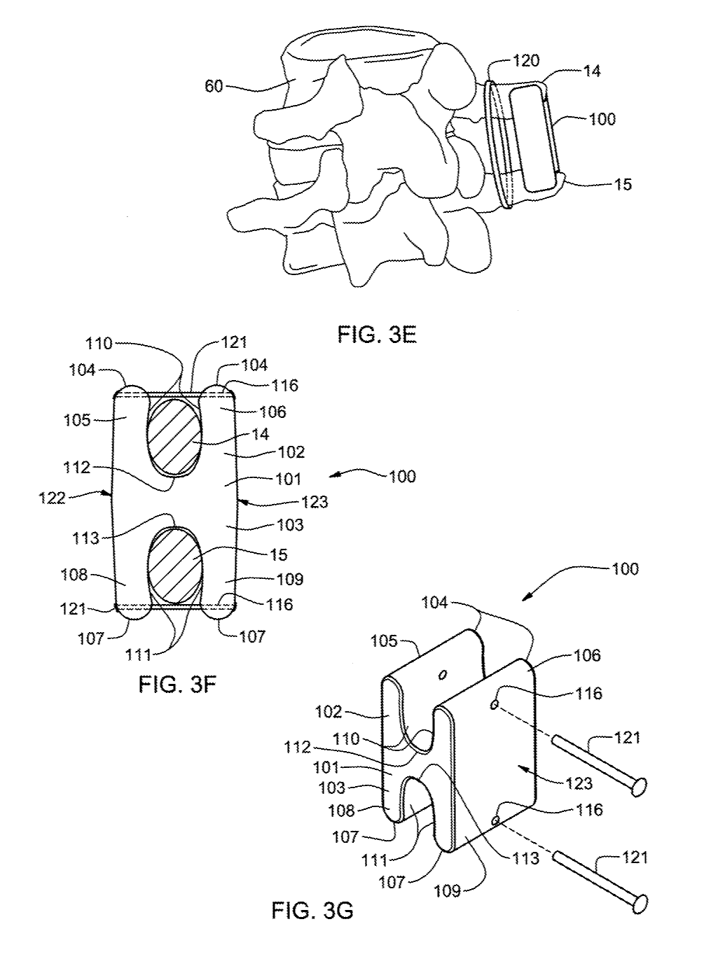

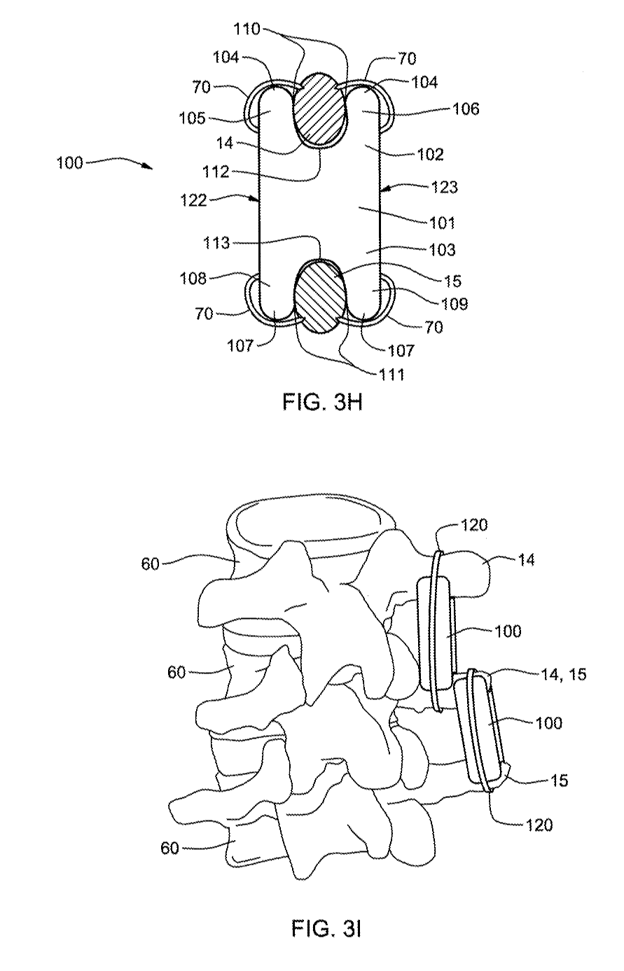

FIG. 3A depicts another alternative embodiment of an inter-transverse process spacer device 100, in accordance with an aspect of the present invention. Inter-transverse process spacer device 100 includes a spacer member 101 comprising a superior end 102 and an inferior end 103 with a central axis (not shown) extending between superior end 102 and inferior end 103. Extending in an upward direction from superior end 102 is one pair of arms 104 that may include an anterior ann 105 and a posterior ann 106.

Extending in a downward direction from inferior end 103 is one pair of arms 107 that may include an anterior arm 108 and a posterior arm 109. Each pair of arms 104, 107 are integral to spacer member 101 and are sized to resist dislodgement of inter-transverse process spacer device 100 following placement between two adjacent transverse processes 14, 15. Further, each pair of arms 104, 107 are centered about the central axis of spacer member 101 resulting in a roughly H-shaped overall structure. An upper U-shaped channel 110 is typically defined by a seat 112, anterior arm 105 and posterior arm 106 and is appropriately sized to receive transverse process 14. Additionally, a lower U-shaped channel 111 is defined by a seat 113, anterior arm 108 and posterior arm 109 and is also appropriately sized to receive transverse process 15. Anterior ann 105 and posterior arm 106 are disposed relatively parallel to each other and project in an upward manner from seat 112. Anterior arm 108 and posterior ann 109 project in a downward manner from seat 113 and are substantially parallel to each other. When in use, inter-transverse process spacer device 100 is maneuvered in a manner allowing two adjacent transverse processes 14, 15 to be positioned within channels 110, 111, causing the anterior aspect of two adjacent transverse processes 14, 15 to contact anterior arms 105, 108 and the posterior aspect of two adjacent transverse processes 14, 15 to contact posterior aims 106, 109.

FIGS. 3B, 3C, 3D, 3F, 3G & 3H show several methods used for securing inter-transverse process spacer device 100 between two adjacent transverse processes 14, 15.

As depicted in FIG. 3B, at least one hole 114 extends from an anterior surface 122 of spacer member 101 to a posterior surface 123. In one approach, a connector 120 is passed through hole 114 following the placement of inter-transverse process spacer device 100 between two adjacent transverse processes 14, 15 and then wraps over the superior aspect of superior positioned transverse process 14. A second connector 120 may be inserted through a second substantially parallel hole 114 that also extends from anterior surface 122 to posterior surface 114, and then wraps over the inferior aspect of a second inferior positioned transverse process 15. The ends of connector 120 may be secured using crimps, knots, ties or other suitable fasteners. It should be understood to those skilled in the art that other connector securement techniques and configurations are contemplated and will depend on the type of connector 120 used.

As illustrated in FIG. 3C, an alternative method for securing inter-transverse process spacer device 100 between two adjacent transverse processes 14, 15 may include extending at least one connector 120 around the entire circumference of the exterior surface of inter-transverse process spacer device 100 and the two adjacent transverse processes 14, 15. As described previously, the ends of connector 120 may then be secured using crimps, knots, ties or other suitable fasteners, although it should be understood to those skilled in the art that other connector end securement techniques and configurations are contemplated and will likely depend on the type of connector 120 utilized post-implantation.

As seen in FIG. 3D, yet another alternative method for securing inter-transverse process spacer device 100 between two adjacent transverse processes 14, 15 is contemplated. FIG. 3D depicts the use of at least one connector 120 in a generally figure-8 configuration. Single or multiple connectors 120 may be inserted through an angled passage or hole 115 that extends from anterior surface 122 of spacer member 101 to posterior surface 123 of spacer member 101 and then is looped over the superior surface of superior positioned transverse process 14 which is seated within upper channel 110. Connector 120 may be further passed through a second angled passage or hole 115 that extends from anterior surface 122 to posterior surface 123 allowing connector 120 to also loop over the inferior surface of inferior positioned transverse process 15 which is cradled within lower channel 111. The two ends of connector 120 may be secured using crimps, knots, ties or other suitable fastener. It should be understood to those skilled in the art that other securement techniques and configurations are contemplated and will depend on the type of connector 120 used.

FIG. 3E shows an alternative use of connector 120 in conjunction with inter-transverse process spacer device 100, wherein connector 120 is positioned offset from inter-transverse process spacer device 100. Connector 120 is wrapped around at least two transverse processes 14, 15 applying a compressive load to the transverse processes. In addition, located between transverse processes 14, 15 is inter-transverse processes spacer device 100 that is positioned between connector 120 and adjacent ligamentis soft tissue structures (not shown) that usually attaches to the lateral aspect or ends of the transverse processes.

Connector 120 may be, for example, in the form of a suture, wire, cable, tether, belt, band, cord or other suitable structure and may be fabricated from a material selected from the group consisting of polyester, polyethlylene, titanium, stainless steel, carbon fiber composite polymers, bio-compatible metals, resorbable polymers, bio-inert polymeric materials, and any combinations of these materials.

Yet a further alternative method for securing inter-transverse process spacer device 100 between two adjacent transverse processes is seen at FIGS. 3F & 3G. As shown, at least one through hole 116 is directed in an anterior to posterior direction and passes through anterior arms 105, 108 and posterior arms 106, 109 located within superior pair of arms 104 and inferior pair of anus 107, respectively. Hole 116 extends through superior pair of arms 104 and is usually substantially parallel to a second hole 116 extending through inferior pair of arm 107. In use, inter-transverse process spacer device 100 is placed between two adjacent transverse processes 14, 15 allowing two adjacent transverse processes 14, 15 to be positioned within upper and lower channels 110, 111. Following final placement of inter-transverse process spacer device 100, one connector 121 may be inserted through hole 116 that is located in the most upper portion of superior pair of arms 104 and will span upper channel 110 across the superior aspect of transverse process 14. Typically, a second connector 121 is inserted through a second hole 116 located in the most downward portion of inferior set of arms 107 and will span lower channel 111 and across the inferior aspect of transverse process 15. The ends of two connectors 121 may be secured using crimps, caps, nuts, rivets, or other suitable fastener devices. It should be understood to those skilled in the art that other securement techniques and configurations are contemplated and will depend on the type of connector 121 used. Connector 121 may be, for example, in the form of a bolt, screw, lock pin, rivet, staple, press-fit pin or other suitable structure for securement between transverse processes 14, 15 and may be fabricated from a material selected from the group consisting of carbon fiber composite polymers, bio-compatible metals, resorbable polymers, bio-inert polymeric materials, and any combinations of these materials.

FIG. 3H depicts yet a further alternative method for securing inter-transverse process spacer device 100 between two adjacent transverse processes 14, 15. Tong structure 70 may be disposed at superior end 102 and inferior end 103 with tong structure 70 being generally ring-shaped and functioning to pierce the anterior and posterior surfaces of transverse processes 14, 15. Alternatively, tong structure 70 may fix inter-transverse process spacer device 100 between two adjacent transverse processes 14, 15 by piercing surrounding soft tissue or ligamentus structures. Tong structure 70 may be fabricated from a deformable or springy material including, but not limited to PEEK, titanium, stainless steel, other bio-compatible metals, other bio-inert polymeric materials and any combinations of these materials.

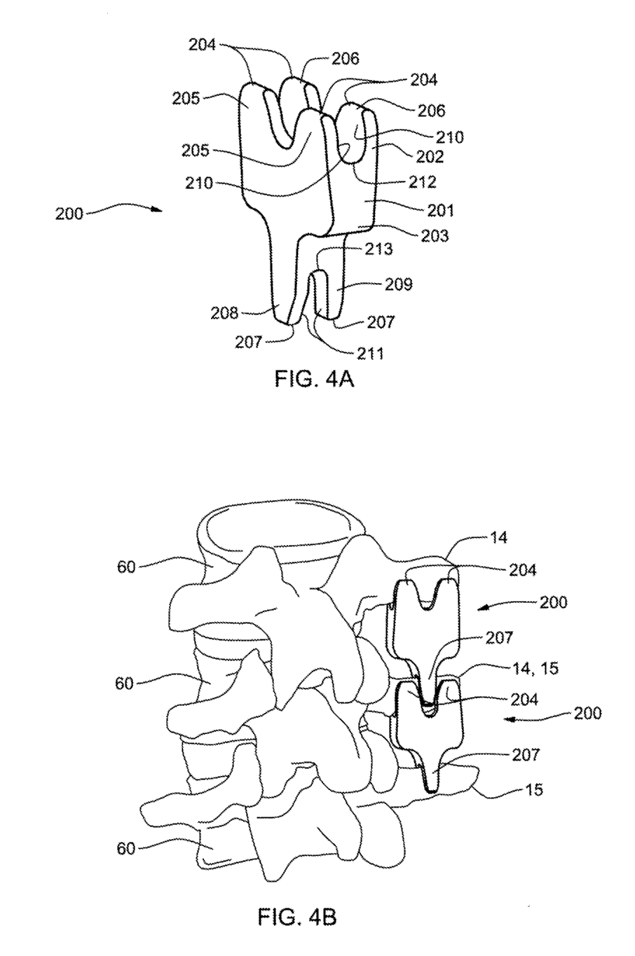

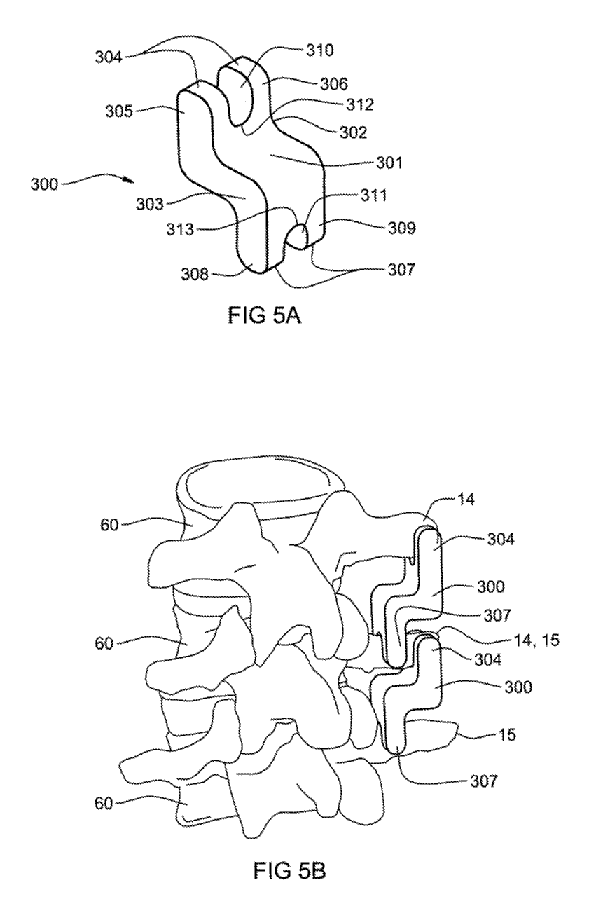

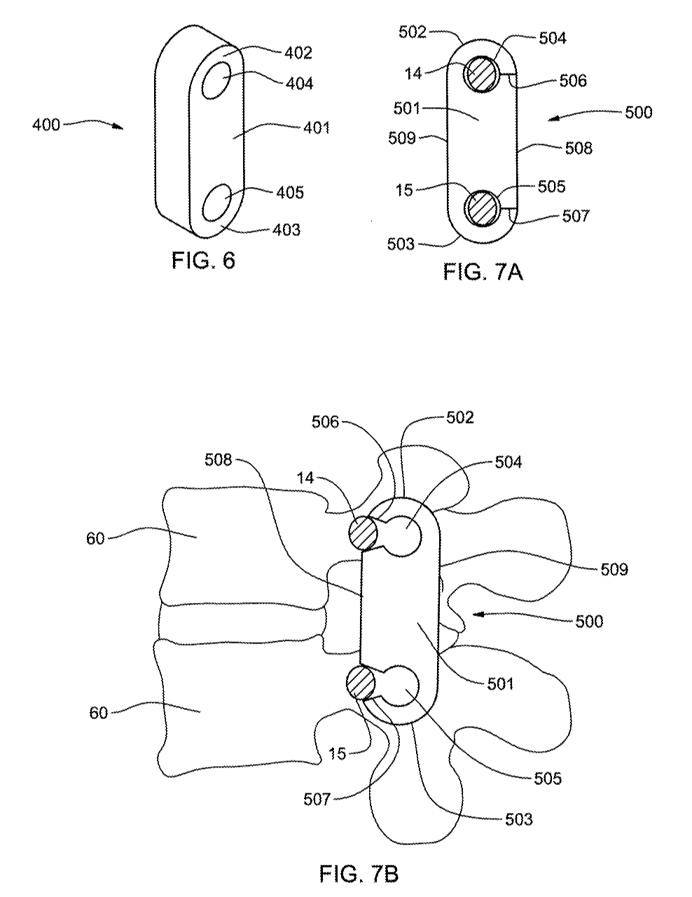

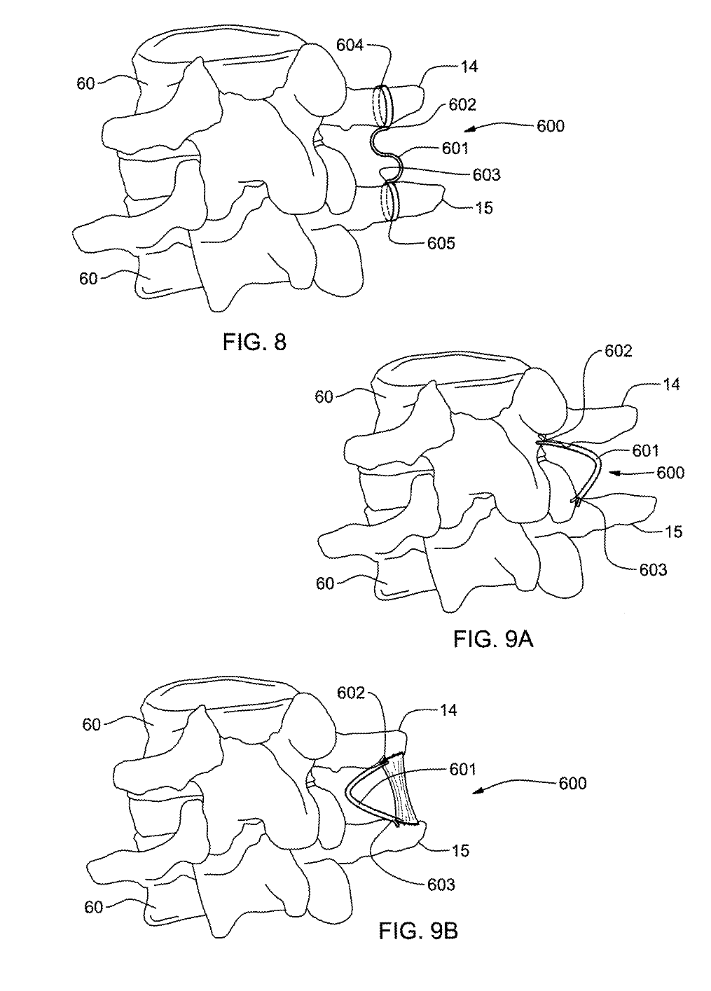

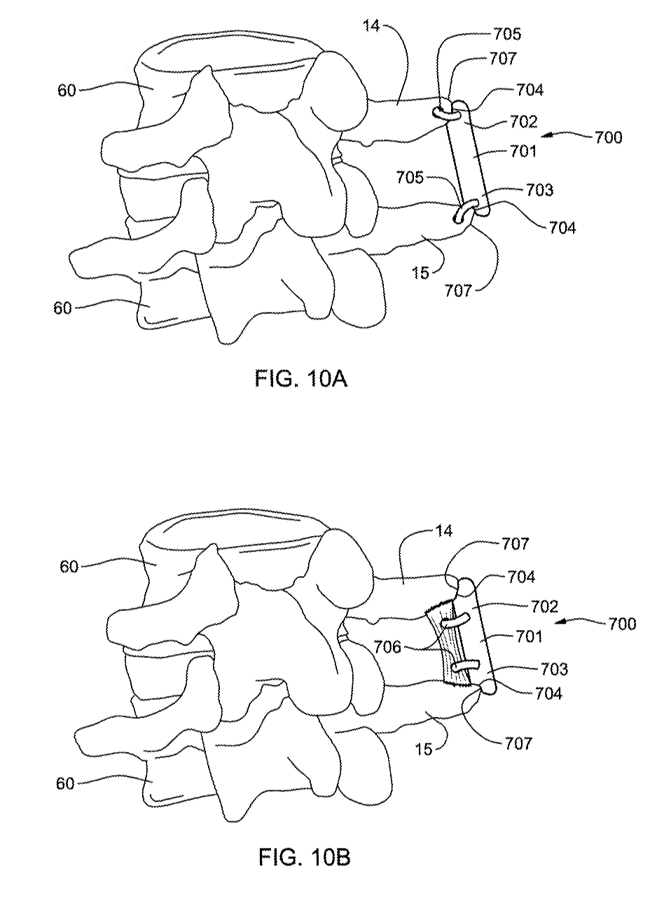

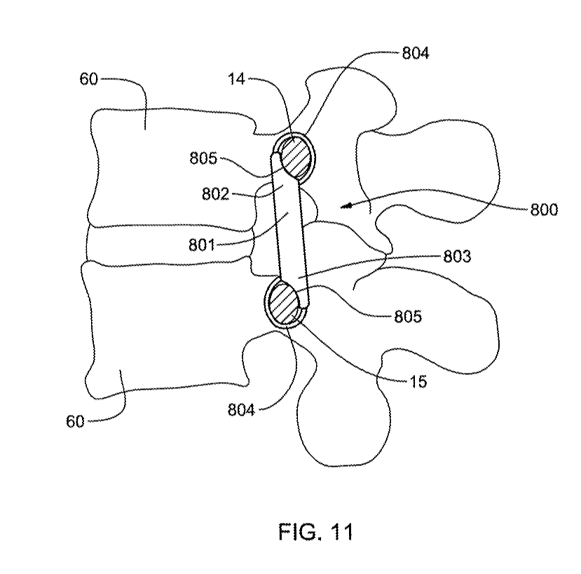

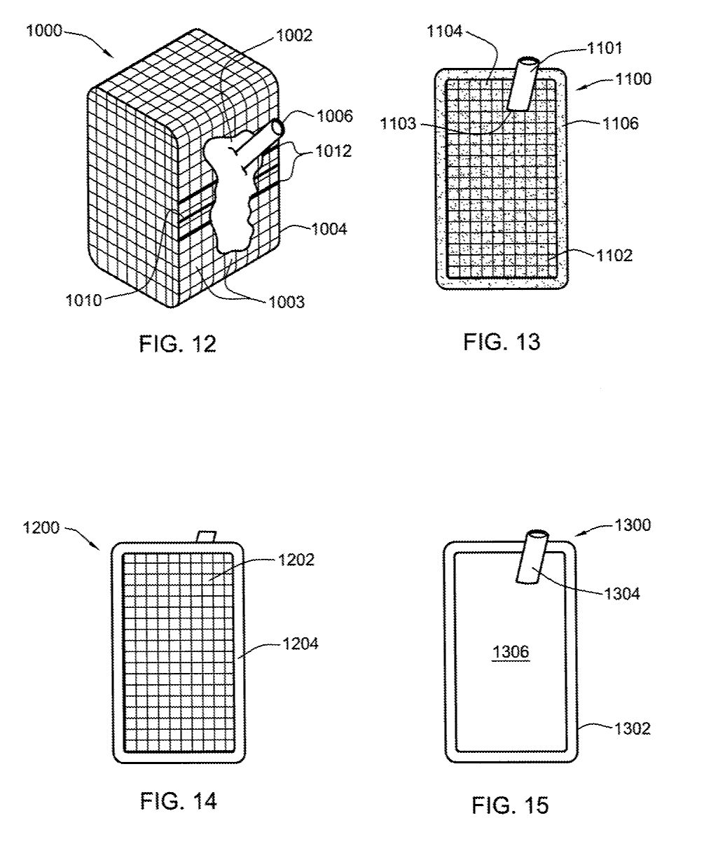

FIG. 3I depicts an inter-transverse process spacer device system that includes a plurality of inter-transverse process spacer devices 100. Multiple inter-transverse process spacer devices 100 are inserted between adjacent transverse processes of several adjacent vertebral bodies 60 at corresponding deformed spinal levels. Adjacent inter-transverse process spacer devices 100 are typically implanted in a stacked manner relative to each other, resulting in a generally overall linear arrangement. As described herein, each of the plurality of inter-transverse process spacer devices 100 may be secured to the transverse process by at least one connector 120, 121 or tong structure 70 (not shown). Alternatively, at least one connector 120 or tong structure 70 may link or couple each of the plurality of inter-transverse process spacer devices 100 to each other (not shown). Typically, the number of inter-transverse process spacer devices 100 implanted will depend upon the severity of the spinal deformity to be corrected and the affected levels of the spinal column. By way of example only, in FIG. 31, two inter-transverse process spacer devices 100 are shown to be placed on the concave side of a medial-lateral deformity.

FIG. 4A depicts another alternative embodiment of an inter-transverse process spacer device 200, in accordance with an aspect of the present invention that includes a spacer member 201 with a superior end 202 and an inferior end 203 with a central axis (not shown) extending between superior end 202 and inferior end 203. Extending in an upward direction from superior end 202 are two pair of arms 204, with each pair of arms including an anterior arm 205 and a posterior arm 206. Extending from inferior end 203 in a downward direction is one pair of arms 207 that may include an anterior arm 208 and a posterior arm 209. Each pair of anus 204, 207 are integral to spacer member 201 usually with one of the two superior pair of arms 204 being offset laterally relative to the central axis and the second of the two superior pair of arms 204 being offset medially relative to the central axis. The inferior pair of arms 207 are centered generally about the central axis resulting in a roughly Y-shaped overall structure defining inter-transverse process spacer device 200.

For each of superior pair of arms 204, an upper channel 210 is typically defined by a seat 212, anterior arm 205 and posterior arm 206. Additionally, for inferior pair of arms 207, a lower channel 211 is defined by a seat 213, anterior arm 208 and posterior arm 209. For both superior pair of arms 204, anterior arm 205 and posterior arm 206 are oriented relatively parallel to each other and project in a generally upward manner from seat 212. For inferior pair of arms 207, anterior arm 208 and posterior arm 209 project in a generally inferior or downward manner from seat 213 and are substantially parallel to each other. Each pair of arms 204, 207, together with seats 212, 213 fonn U-shaped channels 210, 211 respectively, which are each appropriately sized to receive a transverse process 14, 15 and allow inter-transverse process spacer device 200 to resist movement following implantation adjacent to a patient's spinal column.

Although not shown, it is contemplated that either connectors 120, 121 or tong structure 70 may be utilized to secure inter-transverse process spacer device 200 between adjacent transverse processes 14, 15. As described herein, it should be understood to those skilled in the art that connector 120 may pass through anterior to posterior directed, single or multiple, straight or angled holes or passages (not shown) within spacer member 201, thereby allowing connector 120 to wrap or loop around or over both superior pair of arms 204 and inferior pair of arms 207 allowing for securement of inter-transverse process spacer device 200 between adjacent transverse processes 14, 15 in the same or similar manner as described above for inter-transverse process spacer device 100. Further, as discussed herein, it should be understood by those skilled in the art that connector 121 may be inserted through anterior to posterior directed, single or multiple straight holes or passages (not shown) within both superior pair of arms 204 and inferior pair of arms 207. The holes located in both superior pair of arms 204 being substantially parallel to the hole or passage located in inferior pair of arms 207. When in use, connector 121 will be inserted through the holes that are located in the upper most portion of both superior pair of arms 204 spanning each upper channel 210 and the superior aspect of transverse process 14. Additionally, a second connector 121 may be inserted through a hole or passage located in the downward most portion of inferior set of arms 207 spanning lower channel 211 and crossing over the inferior aspect of transverse process 15. Also, it should be understood to those skilled in the art that tong structure 70 may be disposed at the superior end of both superior pairs of arms 204 and the inferior end of inferior pair of arms 207 to secure the inter-transverse process spacer device 200 in place in the same manner that has been previously described herein.

As shown in FIG. 4B, inter-transverse process spacer device 200 is typically placed adjacent to a patient's spinal column between two transverse processes 14, 15. Inter-transverse process spacer device 200 is manipulated in a manner allowing two adjacent transverse processes 14, 15 to be positioned within two upper channels 210 and lower channel 211, causing the anterior aspect of two adjacent transverse processes 14, 15 to contact anterior arms 205, 208 and the posterior aspect of two adjacent transverse processes 14, 15 to contact posterior arms 206, 209.

FIG. 4B also further depicts an alternative embodiment of an inter-transverse process spacer device system that includes a plurality of inter-transverse process spacer devices 200. Multiple inter-transverse process spacer devices 200 are placed between adjacent transverse processes of several vertebral bodies 60 that correspond to the spinal levels of the deformity. Adjacent inter-transverse process spacer devices 200 are implanted in close association relative to each other, resulting in a generally overall linear arrangement of the system. When implanted, the shape and size of inter-transverse process spacer device 200 allows for inferior pair of arms 207 of a superiorly placed inter-transverse process spacer device 200 to be positioned proximate or within the space defined between the two superior pair of arms 204 of an adjacent inferiorly placed inter-transverse process spacer device 200. As described herein, each of the plurality of inter-transverse process spacer devices 200 may be secured between two transverse processes with at least one connector 120, 121 or tong structure 70 (not shown). Alternatively, at least one connector 120 or tong structure 70 may link or couple each of the plurality of inter-transverse process spacer devices 200 to each other (not shown). Typically, the number of inter-transverse process spacer devices 200 implanted is dependent upon the severity of the spinal deformity and the affected levels of the spinal column. By way of example only, in FIG. 4B, two inter-transverse process spacer devices 200 are shown to be used to correct a spinal deformity that spans three levels of the spinal column.

FIG. 5A depicts still another alternative embodiment of an inter-transverse process spacer device 300, in accordance with an aspect of the present invention. Inter-transverse process spacer device 300 includes a spacer member 301 comprising a superior end 302 and an inferior end 303 with a central axis (not shown) extending between superior end 302 and inferior end 303. Extending in an upward direction from superior end 302 is one pair of arms 304 including an anterior arm 305 and a posterior arm 306. Further, extending in a downward direction from inferior end 303 is one pair of arms 307 that may include an anterior arm 308 and a posterior arm 309. Each pair of arms 304, 307 are integral to spacer member 301, usually with superior pair of arms 304 being offset laterally relative to the central axis and inferior pair of arms 307 being offset medially relative to the central axis. It should be understood to those skilled in the art, that an alternative configuration of inter-transverse process spacer device 300 may include each pair of arms 304, 307 to be opposite as described herein, for example, superior pair of arms 304 being offset medially relative to the central axis and inferior pair of arms 307 being offset laterally relative to the central axis. An upper U-shaped channel 310 is typically defined by a seat 312, anterior arm 305 and posterior arm 306 and is sized to receive transverse process 14. Additionally, for inferior pair of arms 307, a lower U-shaped channel 311 is defined by a seat 313, anterior arm 308 and posterior arm 309 and is also sized to receive transverse process 15. Anterior arm 305 and posterior arm 306 are disposed relatively parallel to each other and project in a generally superior direction from seat 312. Inferior pair of arms 307, anterior arm 308 and posterior arm 309 project in a generally inferior direction from seat 313 and are substantially parallel to each other.

Although not shown, as described herein, it is contemplated that either connector 120, 121 or tong structure 70 may be utilized to secure inter-transverse process spacer device 300 between two adjacent transverse processes 14, 15. It should be understood to those skilled in the art that connector 120 may be positioned through anterior to posterior directed, single or multiple, straight or angled holes (not shown) within spacer member 301, thereby allowing connector 120 to wrap or loop around or over superior pair of arms 304 and inferior pair of arms 307 allowing for securement of inter-transverse process spacer device 300 between two adjacent transverse processes 14, 15 in the same or similar manner as described for inter-transverse process spacer device 100. Further, as discussed herein, it should be understood to those skilled in the art that connector 121 may be inserted through anterior to posterior directed, single or multiple straight holes or passages (not shown) within superior pair of arms 304 and inferior pair of arms 307. The hole or passage located in superior pair of arms 304 being substantially parallel to the hole located in inferior pair of arms 307. When in use, connector 121 will be inserted through the hole or passage that is located in the upper most portion of superior pair of arms 304 and span upper channel 310, crossing the superior aspect of transverse process 14. Additionally, a second connector 121 may be inserted through a hole or passage located in the downward most portion of inferior set of arms 307 and span lower channel 311, crossing the inferior aspect of transverse process 15. Also, it should be understood to those skilled in the art that tong structure 70 may be disposed at the superior end of the superior pair of arms 304 and inferior end of inferior pair of arms 307 to secure inter-transverse process spacer device 300 between the two adjacent transverse processes 14, 15.

As illustrated in FIG. 5B, when in use, the inter-transverse process spacer device 300 is proximate to the patient's spinal column. Inter-transverse process spacer device 300 is usually maneuvered in a manner allowing two adjacent transverse processes 14, 15 to be positioned within each of the upper channel 310 and lower channel 311, causing the anterior aspect of two adjacent transverse process 14, 15 to contact anterior arms 305, 308 and the posterior aspect of two adjacent transverse processes 14, 15 to contact posterior anus 306, 309. Upper channel 310 and lower channel 311 are sized and configured to provide resistance to post-operative in vivo forces and stabilize inter-transverse process spacer 300.

FIG. 5B also depicts an alternative embodiment of an inter-transverse process spacer device system which includes multiple inter-transverse process spacer devices 300 implanted adjacent to vertebral bodies 60 at corresponding affected spinal levels. The plurality of inter-transverse process spacer devices 300 are positioned in close approximation relative to each other, resulting in a generally overall linear arrangement of the system. When implanted, the shape and size of inter-transverse process spacer device 300 allows for inferior pair of arms 307 of superior placed inter-transverse process spacer device 300 to either contact or be positioned proximate to spacer member 301 of the adjacent and inferior placed inter-transverse process spacer device 300. Additionally, when implanted, superior pair of arms 304 of inferior placed inter-transverse process spacer device 300 will contact or be in close approximation to spacer member 301 of adjacent superior positioned inter-transverse process spacer device 300. As shown in FIG. 5B, following implantation, transverse process 14 may be simultaneously located within lower channel 311 of a superior placed inter-transverse process spacer device 300 and upper channel 310 of an inferior placed inter-transverse process spacer device 300. As described herein, each of the plurality of inter-transverse process spacer devices 300 may be secured with at least one connector 120, 121 or tong structure 70 (not shown). Alternatively, at least one connector 120 or tong structure 70 may link or couple each of the plurality of inter-transverse process spacer devices 300 to each other (not shown). Usually, the number of inter-transverse process spacer devices 300 implanted is dependent upon the severity of the spinal deformity and the affected levels of the spinal column. By way of example only, in FIG. 5B, two inter-transverse process spacer devices 300 are shown to correct a spinal deformity that spans three levels of the spinal column.

FIG. 6 depicts yet another alternative embodiment of an inter-transverse process spacer device 400, in accordance with an aspect of the present invention, that includes a spacer member 401 having a superior end 402 and an inferior end 403 with a central axis (not shown) extending between superior end 402 and inferior end 403. At least one superior positioned hole 404 extends in a medial to lateral direction passing through spacer member 401 near superior end 402 along the central axis. Further, a second inferior positioned hole 405, extends in a medial to lateral direction and passes through spacer member 401 proximate to inferior end 403. Inferior hole 405 is also aligned along the central axis. Superior hole 404 and inferior hole 405 are usually substantially parallel relative to each other. In use, inter-transverse process spacer device 400 is typically maneuvered in a manner to allow transverse processes 14, 15 to be inserted into superior hole 404 and inferior hole 405, respectively.

Although not shown, an alternative embodiment of an inter-transverse process spacer system includes multiple spacer members 401 being inserted over the transverse processes of several adjacent vertebral bodies 60. As described herein, the transverse processes of the adjacent vertebral bodies 60 are slid into either superior hole 404 or inferior hole 405 depending upon the position of the multiple stacked inter-transverse process spacer devices 400. The stacked arrangement results in a dynamic distraction force being applied of the spinal column to correct the presented deformity.

Another alternative embodiment of an inter-transverse process spacer device 500, in accordance with an aspect of the present invention is shown at FIG. 7A. This embodiment includes a spacer member 501 with a superior end 502 and an inferior end 503 with a central axis (not shown) extending between superior end 502 and inferior end 503. Located along the central axis proximate to the superior end is a superior through hole 504 extending in a medial to lateral direction. A second inferior positioned through hole 505 is also located along the central axis and extends in a medial to lateral direction. Superior hole 504 and inferior hole 505 are usually substantially parallel to each other. Further, a slit 506, extends from an external anterior surface 508 of spacer member 501 and intersects superior hole 504. A second slit 507 also extends from an external anterior surface 508 of spacer member 501 to inferior hole 505. It should be understood to those skilled in the art that it is contemplated that slits 506, 507 may also extend from an external posterior surface 509 of spacer member 501 and intersect superior hole 504 and inferior hole 505 as an alternative to the mirror image embodiment of inter-transverse process spacer device 500 described above.

FIG. 7B shows inter-transverse process spacer device 500 being implanted over two adjacent transverse processes 14, 15. As illustrated, slits 506, 507 are widened to allow transverse processes 14, 15 to slide into superior hole 504 and inferior hole 505. The implantation procedure may occur from either a posterior direction or anterior direction depending upon the location of slits 506, 507. Following final placement of inter-transverse process spacer device 500, slits 506, 507 will close, thereby causing superior hole 504 and inferior hole 565 to surround transverse processes 14, 15 and secure inter-transverse process spacer device 500 in place.

FIG. 8 shows a further alternative embodiment of an inter-transverse process spacer device 600, in accordance with an aspect of the present invention, that includes a spacer member 601, a superior end 602, and an inferior end 603. Attached to superior end 602 may be a superior cuff or ring structure 604 that is configured to slide onto or engage with the superior positioned transverse process 14. Attached to inferior end 603 is a second inferior cuff or ring structure 605 that is also configured to attach or slidingly engage an inferior positioned transverse process 15. Spacer member 601 may be a coil or spring-like structure that is sized to be inserted between adjacent transverse processes 14, 15.

As illustrated in FIGS. 9A & 9B, spacer member 601 may also be a “bow” spring-like structure that can be inserted between the two transverse processes 14, 15 with the bow apex oriented either laterally or medially. Superior end 602 and inferior end 603 may be V-shaped or wishbone shaped structures for ease of engaging transverse processes 14, 15. When the bow apex is directed laterally, the V-shaped superior end 602 and inferior end 603 may engage transverse processes 14, 15 at the junction of the transverse process and pedicle notch of the vertebral body 60. (See FIG. 9A). Alternatively, when the bow apex is directed medially the V-shaped superior end 602 and inferior end 603 will engage the lateral attachment site of the lateral ligamentus structure and corresponding transverse process. (See FIG. 9B.) The two geometric constructs of spacer member 601 are of an appropriate stiffness and are manufactured from a material, for example, PEEK or titanium, to produce a resultant dynamic force sufficient enough to correct a spinal deformity following implantation.

Although not shown, multiple inter-transverse process spacer devices 600 may be inserted between several adjacent transverse processes to comprise an alternative inter-transverse process spacer device system. The plurality of inter-transverse process spacer devices 600 will be used in a serial arrangement to dynamically produce a force large enough to correct a spinal deformity. The multiple inter-transverse process spacer devices 600 will usually be positioned on the concave side of the deformity, thereby producing a distraction force in an attempt to straighten and correct the spinal deformity.

FIG. 10A depicts yet another alternative embodiment of inter-transverse process spacer device 700, in accordance with an aspect of the present invention. Inter-transverse process spacer device 700 includes a spacer member 701 with a superior end 702 and an inferior end 703. Superior end 702 and inferior end 703 are configured with generally cup-shaped or concave surface regions 704 to facilitate close contact with the lateral end 707 of two adjacent transverse processes 14, 15. When in use, superior end 702 and inferior end 703 of inter-transverse process spacer device 700 are positioned adjacent to lateral ends 707 of transverse processes 14, 15. Lateral ends 707 engage concave surface regions 704 with spacer member 701 positioned between transverse processes 14, 15. The inter-transverse process spacer device 700 is secured to transverse processes 14, 15 by a tether-like structure 705 that is attached to the anterior and posterior sides of superior end 702 and inferior end 703 and passes through an anterior to posterior directed hole drilled through the transverse processes 14, 15. Alternatively, tether-like structure 705 may attach only to the anterior cortex and/or posterior cortex of two adjacent transverse processes 14, 15.

An alternative method for securing inter-transverse process spacer device 700 is seen at FIG. 10B. When in final position, concave surface regions 704 again contact lateral ends 707 with at least one tether 706 being coupled to the lateral ligamentus structure that connects the two adjacent transverse processes 14, 15. Tether 706 usually will be threaded through the ligamentus structure to ensure rigid fixation of spacer member 701 between the two adjacent transverse processes 14, 15.

It should be understood to those skilled in the art that other securemeut techniques and configurations are contemplated and will depend upon the type of tether-like structure 705, 706 that is used. For example only, tethers 705, 706 may be in the form of a suture, wire, cable, band, cord, or other suitable structure and may be fabricated from a material selected from the group consisting of polyester, polyethylene, titanium, stainless steel, carbon fiber composite polymers, bio-compatible metals, resorbable polymers, bio-inert polymeric materials and any combination of these materials.

A further alternative embodiment of an inter-transverse process spacer device 800, in accordance with an aspect of the present invention comprises a spacer member 801, a superior end 802 and an inferior end 803. FIG. 11 shows superior end 802 and inferior end 803 both being shaped and configured with a curved surface or angled bias 805. The angled bias 805 contacts the outer cortex of adjacent transverse processes 14, 15. Superior end 802 of inter-transverse process spacer device 800 is held in place by tether mechanism 804 that attaches to one end of the angled bias 805, loops over the superior aspect of the superior positioned transverse process 14 and attaches to a second end of angled bias aspect 805. The same tether mechanism 804 and angled bias 805 is used to secure inferior end 803 to the transverse process 15. The tether mechanism 804 may secure angled bias 805 to either the anterior side of the transverse process or the posterior side of transverse processes 14, 15 depending on the positioning of space member 801. As seen in FIG. 11, angled bias 805 of superior end 802 is positioned on the anterior side of transverse process 14 and angled bias 805 of inferior end 803 is positioned on the posterior side of transverse process 15. It should be understood to those skilled in the art that inter-transverse process spacer device 800 may also be fabricated as a mirror image of the device described above herein. For example only, tether mechanism 804 may be in the fonn of a suture, wire, cable, belt, band, cord or other suitable structure and may be fabricated from a material selected from a group consisting of polyester, polyethylene, titanium, stainless steel, carbon fiber composite polymers, bio-compatible metals, resorbable polymers, bio-inert polymeric materials, elastic materials and any combination of these materials.

Although not shown, an alternative inter-transverse process spacer device system may be comprised of either a plurality of inter-transverse process spacer devices 700 or a plurality of inter-transverse process spacer devices 800. As described previously herein, each of the plurality of inter-transverse process spacer devices 700, 800 may be secured to each of the transverse processes using at least one tether 705, 706 or tether mechanism 804, respectively. Additionally, it is contemplated that tether 705, 706 may link or couple each of the plurality of inter-transverse process spacer devices 700 to each other. Also, tether mechanism 804 may connect or couple each of the individual inter-transverse process spacer devices 800 together. As described herein, the number of inter-transverse process spacer devices 700, 800 that are used intra-operatively will depend directly upon the severity of the spinal deformity and the number of affected levels in the spinal colunm.

With respect to the various embodiments of the inter-transverse process spacer device 10, 50, 100, 200, 300, 400, 500, 600, 700 and 800 described herein, the inter-transverse process spacer device 10, 50, 100, 200, 300, 400, 500, 600, 700 and 800 may be fabricated from materials that are flexible or exhibit at least some flexibility and deformability. Additionally, the construct materials may be resilient and/or elastic, so the corresponding spacer members can assume various shapes during and after insertion and securement between two adjacent transverse processes 14, 15.

The inter-transverse process spacer device 10, 50, 100, 200, 300, 400, 500, 600, 700 and 800 may be made from any biocompatible material, material of synthetic or natural origin, and material of a resorbable or non-resorbable nature. Suitable examples of construct materials include resorbable materials including polylactide, polyglycolide, tyrosine-derived polycarbonate, polyanhydride, polyorthoester, polyphosphazene, calcium phosphate, hydroxyapatite, bioactive glass, collagen, albumin, fibrinogen and combinations thereof; and non-resorbable materials including polyethylene, polyester, polyvinyl alcohol, polyacrylonitrile, polyamide, polytetrafluorethylene, poly-paraphenylene terephthalamide, polyetheretherketone, polyurethane, and combinations thereof. Further, non-resorbable materials may include carbon-reinforced polymer composites, shape-memoiy alloys, titanium, titanium alloys, cobalt chrome alloys, stainless steel, and combinations thereof. The inter-transverse process spacer device 10, 50, 100, 200, 300, 400, 500, 600, 700 and 800 is fabricated from a material capable of resisting compressive motion (or loads) with a stiffness of about 10 to about 3000 N/mm (newtons per millimeter).

FIGS. 12, 13, 14 & 15 show a further alternative embodiment of the inter-transverse process spacer device 1000, in accordance with an aspect of the present invention, that can be formed in situ during a surgical procedure. Inter-transverse process spacer device 1000 includes the following basic aspects: a flexible container 1002 and a structure 1004 for at least part of flexible container 1002 that controls at least part of the shape of inter-transverse process spacer device 1000. Flexible container 1002 can be filled or injected through optional conduit 1006 after placement. Further, structure 1004 may be folded or otherwise reduced in size prior to use in some aspects. Together with an unfilled container 1002, in some aspects, inter-transverse process spacer device 1000 can create a smaller footprint during implantation. Once filled, structure 1004 provides support and containment for the flexible container 1002, as well as providing shape control for at least part of inter-transverse process spacer device 1000.

FIG. 12 depicts a partially cut-away view of inter-transverse process spacer device 1000. As shown in FIG. 12, inter-transverse process spacer device 1000 comprises an unfilled flexible container 1002 inside structure 1004. Flexible container 1002 is in an evacuated state during implantation and prior to being filled. Where a valve (e.g., a one-way valve) is coupled to flexible container 1002, with flexible container 1002 typically being evacuated prior to or during the process of coupling the valve thereto. In this embodiment, structure 1004 is inside flexible container 1002. However, as will be described in more detail below, flexible container 1002 can be outside structure 1004, or flexible container 1002 and structure 1004 can be integrated. In addition, although structure 1004 is shown to be a generally rectangular unitary body shape to fit between adjacent transverse processes, structure 1004 may have any shape necessary for the particular surgical application. For example, structure 1004 could instead have a roughly H-shape to fit between two transverse processes. As another example, structure 1004 could be spherically or elliptically shaped to provide better support between bony structures. Further, although structure 1004 is shown enveloping the flexible container 1002, structure 1004 could be for only a portion of flexible container 1002, depending on the particular surgical application. For example, it may be desired to prevent bulging of flexible container 1002 only in a particular area. Coupled to flexible container 1002 is optional conduit 1006 for delivering a material that is compressible following implantation. Structure 1004 provides support for and containment of flexible container 1002, when filled.

Flexible container 1002 is usually flexible and substantially impermeable to the material it will be filled with. However, depending on the application, flexible container 1002 may be permeable to other materials, for example, it may be air and/or water penneable. In the present example, flexible container 1002 takes the form of bag or balloon, but can take other forms, so long as it is flexible and substantially impermeable to the material it will be filled with. Thus, flexible container 1002 must be substantially impermeable to the injectable material, for example, in a liquid state during filling and prior to curing. Examples of container materials include silicone, rubber, polyurethane, polyethylene terephthalate (PET), polyolefin, polycarbonate urethane, and silicone copolymers.

Conduit 1006 usually delivers the injectable material being used to fill flexible container 1002. Conduit 1006 comprises a one-way valve, however, a two-way valve is also contemplated, as another example. Conduit 1006 can comprise any material suitable for implanting, for example, various plastics. Also, conduit 1006 is constructed to be used with a delivery system for filling flexible container 1002, such as, for example, a pressurized syringe-type delivery system. However, the delivery system itself forms no part of the present invention. It is contemplated that, conduit 1006 may be optional. Other examples of how to fill flexible container 1002 comprise the use of a self-sealing material for flexible container 1002, or leaving an opening in flexible container 1002 that is closed (e.g., sewn shut) intraoperatively after filling. Using a curable material to fill flexible container 1002 may also serve to self-seal flexible container 1002.

In use, flexible container 1002 is filled with an injectable material that is compressible following implantation between two adjacent transverse processes 14, 15 of a patient. The compressibility characteristic ensures that the injected material exhibits viscoelastic behavior and that, along with structure 1004, the inter-transverse process spacer device 1000 can accept compressive loads. Generally, inter-transverse process spacer device 1000 may be capable of resisting compressive motion (or loads) with a stiffness of about 10 to about 3000 N/mm (newtons per millimeter). The material is usually injectable, and may be compressible immediately or after a time, for example, after curing. For purposes of this invention, the compressibility characteristic is necessary during end use, i.e., after implantation. Materials that could be used include, for example, a plurality of beads (e.g., polymer beads) that in the aggregate are compressible, or materials that change state from exhibiting fluid properties to exhibiting properties of a solid or semi-solid. Examples of such state-changing materials include two-part curing polymers and/or adhesives, for example, platinum-catalyzed silicone, epoxy or polyurethane.

As noted above, structure 1004 provides support for and containment of container 1002 when filled, as well as at least partial shape control of inter-transverse process spacer device 1000. Structure 1004 comprises, for example, a stnictural mesh comprising a plurality of fibers and/or wires 1008. Within the structural mesh are shape-control fibers and/or wires 1010. In one example, shape control is provided by wires of a shape-memory alloy (e.g., Nitinol). Shape-memory alloy wire(s) 1010 can be coupled to the structural mesh (inside or outside), or weaved into the mesh (i.e., integrated). Coupling can be achieved, for example, by stitching, twisting, or closing the wire on itself. Alternatively, shape control can be provided by other wires or fibers that do not “give” in a particular direction, for example, metal or metal alloys (e.g., tantahun, titanium or steel, and non-metals, for example, carbon fiber, PET, polyethylene, polypropylene, etc.). The shape-memory alloy can be passive (e.g., elastic) or active (e.g., body-temperature activated). The use of metal, metal alloy or barium coated wires or fibers can also improve radiopacity for imaging. The remainder of structure 1004 can take the form of, for example, a fabric jacket, as shown in FIG. 12. Although the shape-memory alloy wires 1010 make up only a portion of the structural mesh of FIG. 12, it will be understood that there could be more such wires, up to and including comprising the entirety of the mesh. The fabric jacket in this example contains and helps protect flexible container 1002 from bulging and damage from forces external to flexible container 1002, while the shape-memory alloy provides shape control of inter-transverse process spacer device 1000 in a center region 1012. The fibers of the jacket comprise, for example, PET fabric, polypropylene fabric, polyethylene fabric and/or steel, titanium or other metal wire. Depending on the application, structure 1004 may be permeable to a desired degree. For example, if bone or tissue growth is desired to attach to structure 1004, permeability to the tissue or bone of interest would be appropriate. As another example, penneability of structure 1004 may be desired to allow the material used to fill flexible container 1002 to evacuate air or water, for example, from flexible container 1002, in order to prevent bubbles from forming inside. Where a mesh is used, for example, the degree of penneability desired can be achieved by loosening or tightening the weave.

Although structure 1004 is shown in a unitary rectangular body shape in the example of FIG. 12, it will be understood that in practice, structure 1004 can be made to be folded, unexpanded, or otherwise compacted. This is particularly true where, for example, structure 1004 comprises a fabric or other easily folded material. A folded or unexpanded state facilitates implantation, allowing for a smaller surgical opening, and unfolding or expansion in situ upon filling of flexible container 1002. Further, structure 404 can have a different final shape, depending on the shape-control material used. For example, the shape-memory wires in FIG. 12 may be in their inactive state, whereupon activation by body temperature causes contraction thereof, making the spacer of FIG. 12 “thinner” than shown in center region 1012.

FIG. 13 depicts an outer view of another example of an inter-transverse process spacer device 1100, in accordance with an aspect of the present invention. A flexible container conduit 1101 is shown pointing outward from an opening 1103. As shown, the structure 1102 delimits the final shape of inter-transverse process spacer device 1100. Structure 1102 comprises a mesh 1104 of shape-memory alloy wire, that is soaked through with a dispersion polymer 1106 (e.g., silicone). Dispersion polymer 1106 (after curing) acts as the flexible container and is shown filled in FIG. 13. This is one example of the flexible container and structure 1102 being integral. Although mesh 1104 of FIG. 13 is described as being all shape-memory alloy wire, it will be understood that, like FIG. 12, the shape-memory alloy could only form a part of structure 1102.

FIG. 14 is a cross-sectional view of another example of an inter-transverse process spacer device 1200, in accordance with an aspect of the present invention. Inter-transverse process spacer device 1200 is similar to inter-transverse process spacer device 1100 of FIG. 13, except that instead of being soaked in a dispersion polymer, a structural mesh 1202 of a shape-memory alloy wire is coated with a dispersion polymer (e.g., silicone) 1204 or other curable liquid appropriate for the container material, creating an outer flexible container. The coating can be done in a conventional manner, for example, by dip molding on the outside of the mesh.

FIG. 15 depicts yet another example of an inter-transverse process spacer device 1300 with an integrated flexible container and structure, in accordance with another aspect of the present invention. The flexible container and structure in the example of FIG. 15 both comprise a single layer 1302 of rubber that is thick enough for a given application to perform the functions of both the flexible container and structure (including shape control). Such a rubber shell would be able to return to its original shape when unconstrained. In addition, inter-transverse process spacer device 1300 typically includes a conduit 1304 (preferably, a one-way valve) for filling the internal space 1306. The injectable material can be any of the filling materials described above, for example, silicone.

In an alternate aspect, the rubber shell 1302 of FIG. 15 can be augmented with internal, external, or integrated features to further control shape. Examples of such features include thread, wires (e.g., metal, including shape-memory alloys), cables, tethers, rings or a mesh.

The method for correcting a spinal deformity includes, obtaining at least one inter-transverse process spacer device, the inter-transverse process spacer device 10 includes a spacer member 11 comprising a superior end 12 and an inferior end 13 with a central axis (not shown) extending between superior end 12 and inferior end 13. Spacer member 11 is sized and configured for implantation between two adjacent transverse processes 14, 15. The method further includes positioning inter-transverse process spacer device 10 between two adjacent transverse processes 14, 15. The inter-transverse process spacer device 10 is maneuvered and manipulated in a manner that results in the securement of spacer member 11 adjacent to the spinal column to produce a distraction force or compressive force, depending upon the spinal curvature geometry, for correcting a spinal deformity. It is further understood that the method may include inserting connectors 40, 120, 121 and tong structure 70 into each of the inter-transverse process spacer devices 10 following implantation between the adjacent transverse processes 14, 15. At least one connector 40, 120, 121 and tong structure 70 may be utilized with each individual inter-transverse process spacer device 10, or alternatively, at least one connector 40, 120, 121 and tong structure 70 may link or couple a plurality of inter-transverse process spacer devices 10 to each other. It should be understood to those skilled in the art that the steps of the method for correcting a spinal deformity herein are analogous to those that may be used with the above-described alternative embodiments of inter-transverse process spacer devices 50, 100, 200, 300, 400, 500, 600, 700, and 800.

The method for correcting a spinal deformity utilizing an alternative embodiment of the inter-transverse process spacer device includes, providing at least one inter-transverse process spacer device 1000, the inter-transverse process device 1000 includes a flexible container 1002 used to contain an injectable material, with flexible container 1002 being preferably impermeable to the injectable material, a conduit 1006 coupled to flexible container 1002 for delivering the injectable material and a structure 1004, that controls at least part of flexible container 1002 after injectable material is injected through conduit 1006 and into flexible container 1002. Structure 1004 has a shape that is sized and configured for placement between two adjacent transverse processes of a patient. The method usually provides for inter-transverse process spacer device 1000 to be implanted proximate to the spinal column space between two adjacent transverse processes. The method would also typically include injecting the injectable material preferably through conduit 1006 into flexible container 1002, the injectable material being compressible following inter-transverse process spacer device 1000 implantation between two adjacent transverse processes. The compressibility characteristic ensures that the injectable material exhibits viscoelastic behavior and that, along with structure 1004, the inter-transverse process spacer device 1000 can accept compressive loads and produce distraction forces for correcting a spinal deformity within a patient.

Although the preferred embodiments have been depicted and described in detail herein, it will be apparent to those skilled in the relevant art that various modifications, additions and substitutions can be made without departing from its essence and therefore these are to be considered to be within the scope of the following claims.

What is claimed is:

1. A method for correcting a spinal deformity, the method comprising:
obtaining at least one inter-transverse process spacer device comprising a spacer member having a first end, a second end, and a central axis extending therebetween, wherein the spacer member is sized and configured for placement between a first transverse process and an adjacent second transverse process of a patient; and
positioning the at least one inter-transverse process spacer device between the first transverse process and the adjacent second transverse process of the patient, thereby producing a force for correcting a spinal deformity of the patient.

2. The method of claim 1, wherein the obtaining further comprises fabricating the spacer member from a deformable material.

3. The method of claim 1, wherein the obtaining further comprises providing the spacer member, the spacer member comprising an inner portion and an outer portion, and wherein the inner portion is fabricated from a first deformable material and the outer portion comprises a second deformable material, the inner portion contacting the two adjacent transverse processes when the spacer member is positioned substantially parallel to the patient's spinal column.

4. The method of claim 3, wherein the first deforniable material comprises a first compression modulus and the second deformable material comprises a second compression modulus, wherein the first compression modulus is less than the second compression modulus.

5. The method of claim 4, wherein at least one of the first compression modulus and the second compression modulus has a value ranging from 0.003 to 4.2 GPa.

6. The method of claim 1, wherein the positioning further comprises inserting a second inter-transverse process spacer device between the second transverse process and an adjacent third transverse process, the second end of the at least one inter-transverse process spacer device being proximate to a first end of the second inter-transverse process spacer device, thereby producing a force for correcting a spinal deformity of the patient.

7. The method of claim 1, wherein the obtaining further comprises providing the inter-transverse process spacer device, the inter-transverse process spacer device comprising a first pair of arms extending from the first end of the spacer member and a second pair of anns extending from the second end of the spacer member, wherein the spacer member, first pair of anus and second pair of anus are each centered about the central axis and are configured for placement between two adjacent transverse processes of a patient to dynamically produce a force for correcting a spinal deformity of the patient.

8. The method of claim 7, wherein the first pair of arms includes a first anterior ann and a first posterior arm, the first anterior arm and the fust posterior arm extending substantially parallel to each other and defining a first channel therebetween, and wherein the first channel is sized to receive a first transverse process of the two adjacent transverse processes, and wherein the second pair of anus includes a second anterior ann and a second posterior arm, with the second anterior ann and the second posterior arm extending substantially parallel to each other and defining a second channel therebetween, wherein the second channel is sized to receive a second transverse process of the two adjacent transverse processes of the patient.

9. The method of claim 7 further comprising positioning a first inter-transverse process spacer device between a first transverse process and an adjacent second transverse process and positioning a second inter-transverse process spacer device between the second transverse process and a third adjacent transverse process, wherein the second pair of arms of the first inter-transverse process spacer device and the first pair of arms of the second inter-transverse process spacer device are proximate to each other, thereby producing a dynamic force for collecting a spinal deformity of the patient.

10. The method of claim 7, wherein the obtaining further comprises the inter-transverse process spacer device further comprising one pair of the first pair of arms and the second pair of arms being offset in a lateral direction relative to the central axis of the spacer member, and one pair of the first pair of arms and the second pair of arms being offset in a medial direction relative to the central axis of the spacer member.

11. The method of claim 10 further comprising positioning a first inter-transverse process spacer device between the first transverse process and the adjacent second transverse process of the patient and positioning a second inter-transverse process spacer device between the second transverse process and a third transverse process, and wherein when implanted, the first inter-transverse process spacer device is positioned for close approximation relative to the second inter-transverse process spacer device to produce a force for correcting a spinal deformity of the patient.

12. The method of claim 7, wherein the inter-transverse process spacer further comprises at least one additional pair of anus extending from the first end of the spacer member, the at least one additional pair of arms are configured for engaging a transverse process, wherein the at least one additional pair of arms extending from the first end of the spacer member is offset in the lateral direction relative to the central axis and the first pair of arms extending from the first end of the spacer member is offset in the medial direction relative to the central axis, and wherein the second pair of arms extending from the second end of the spacer member is centered about the central axis.

13. The method of claim 12, further comprising positioning a first inter-transverse process spacer device between the first transverse process and the adjacent second transverse processes and positioning a second inter-transverse process spacer device between the second transverse process and a third adjacent transverse process, wherein the first transverse process spacer device and the second inter-transverse process spacer device are shaped and dimensioned to allow for close association between each other to produce dynamic force for correcting a spinal deformity of the patient.

14. The method of claim 1, wherein the obtaining further comprises providing the spacer member, the spacer member further comprising at least one hole extending therethrough between a medially oriented side and a laterally oriented side of the spacer member when positioned between the two adjacent transverse processes, and wherein at least one of the two adjacent transverse processes extends through the at least one hole when the spacer member is employed to produce a force for correcting the spinal deformity of the patient.

15. The method of claim 14, wherein the positioning further comprises inserting the first transverse process through the at least one hole, and wherein the spacer member comprises multiple holes extending therethrough between a medially oriented side and a laterally oriented side of the spacer member when positioned between the two adjacent transverse processes, and wherein the second transverse process is inserted through a second hole of the multiple holes, thereby positioning the inter-transverse process spacer device substantially parallel to a patient's spinal colunm.

16. The method of claim 14, wherein the obtaining further comprises providing the spacer member, the spacer member further comprising at least one slit extending from an external surface of the spacer member to a first hole of the multiple holes, wherein the at least one slit facilitates positioning at least one of the two transverse processes into the first hole of the multiple holes when positioning the inter-transverse process spacer device substantially parallel to a patient's spinal column.

17. The method of claim 16, wherein the positioning further comprises inserting the first transverse process through the at least one slit and into the first hole of the multiple holes, and wherein the at least one slit is multiple slits, and wherein the second transverse process is inserted through a second slit of the multiple slits into a second hole of the multiple holes, thereby positioning the inter-transverse process spacer device substantially parallel to the patient's spinal column.

18. The method of claim 1, wherein the obtaining further comprises providing the first end of the spacer member, the first end further comprising a first concave surface, and providing the second end of the spacer member, the second end further comprising a second concave surface, and wherein the first concave surface and the (second concave surface are configured to facilitate placement of the spacer member between the two adjacent transverse processes.

19. The method of claim 18, wherein the positioning further comprises coupling a lateral end of the first transverse process to the first concave surface and coupling a lateral end of the second transverse process to the second concave surface, wherein the inter-transverse process spacer device further comprises at least one tether, the at least one tether is multiple tethers, a first tether of the multiple tethers couples the first concave surface to the lateral end of the first transverse process and a second tether of the multiple tethers couples the second concave surface to the lateral end of the second transverse process.

20. The method of claim 1, wherein the obtaining further comprises providing the first end of the spacer member, the first end further comprising a first angled bias surface, and providing the second end of the spacer member, the second end further comprising a second angled bias surface, and wherein the first angled bias surface and the second angled bias surface are configured to facilitate securement of the inter-transverse process spacer device to the first transverse process and the second transverse process.

21. The method of claim 20, wherein the positioning further comprises engaging the first angled bias surface to the first transverse process and engaging the second angled bias surface to the second transverse process, wherein the inter-transverse process spacer device further comprises at least one tether, the at least one tether is multiple tethers, a first tether of the multiple tethers secures the first angled bias surface to the first transverse process and a second tether of the multiple tethers secures the second angled bias surface to the second transverse process.

22. The method of claim 1, wherein the obtaining further comprises providing at least one connector, wherein the at least one connector is configured to facilitate securing the spacer member between the first and second transverse processes of the patient.

23. The method of claim 1, wherein the obtaining further comprises the spacer member, the spacer member further comprising at least one hole extending therethrough between the anteriorly oriented side and the posteriorly oriented side of the spacer member when positioned between the first and second transverse processes and the at least one connector extends through the at least one hole when employed to secure the spacer member to the first and second transverse processes.

24. The method of claim 7, wherein the first pair of arms and the second pair of arms each include at least one hole extending therethrough between an anteriorly oriented side and a posteriorly oriented side of the spacer member when positioned between the first and second transverse processes, with the at least one hole disposed within the first pair of arms extending in a direction substantially parallel to a direction of the at least one hole disposed within the second pair of arms, wherein the at least one connector is multiple connectors, and wherein a first connector of the multiple connectors extends through the at least one hole in the first pair of anns and a second connector of the multiple connectors extends through the at least one hole in the second pair of arms when employed to secure the inter-transverse process spacer device between the first and second transverse processes of the patient.

25. The method of claim 22, wherein the obtaining further comprises providing the spacer member with at least one channel member disposed on at least one of the anteriorly oriented side and posteriorly oriented side of the spacer member when positioned between the first and second transverse processes, the at least one connector extending through the at least one channel member securing the spacer member to the first and second transverse processes of the patient.

26. The method of claim 22, wherein the at least one connector is offset from the inter-transverse process spacer device and is configured to couple the first transverse process to the second transverse process, thereby securing the inter-transverse process spacer device between the first and second transverse processes of the patient.

27. The method of claim 1, wherein obtaining further comprises providing the spacer member, the spacer member comprising a spring structure with the first end and second end securing the spacer member between the two adjacent transverse processes to dynamically produce a force for correcting a spinal deformity of the patient.

28. A method for correcting a spinal deformity, the method comprising:
employing an inter-transverse process spacer device, the inter-transverse process spacer device comprising:
a flexible container for containing an injectable material, the flexible container being substantially impermeable to the injectable material and compressible following implantation between two adjacent transverse processes of a patient;
a conduit coupled to the flexible container for delivering the injectable material into the flexible container; and
a structure associated with the at least part of the flexible container, the structure having a shape of the inter-transverse process spacer device, with the inter-transverse process spacer device being sized and configured for placement between two adjacent transverse processes of a patient;
positioning the inter-transverse process spacer device between the two adjacent tansverse processes of the patient; and
injecting the injectable material into the flexible container through the conduit Thereby expanding the flexible container to the shape of the structure and producing a force for correcting a spinal deformity of the patient.
